(19) United States
(12) Patent Application Publication
(10) Pub. No.: US 2004/0235923 A1
(43) Pub. Date: Nov. 25, 2004

(54) Body temperature-raising agent of amino acids for eating or drinking and for medical use

(76) Inventors: Takashi Abe, Warabi-shi (JP); Hajime Tsunoo, Oota-ku (JP); Masayuki Uchida, Kouza-gun (JP); Takae Wakabayashi, Odawara-shi (JP); Hiroshi Tsuchita, Nishitokyo-shi (JP)

Correspondence Address: SUGHRUE MION, PLLC, 2100 PENNSYLVANIA AVENUE, N.W., SUITE 800, WASHINGTON, DC, 20037, US

(21) Appl. No.: 10/479,605

(22) PCT Filed: Jun. 6, 2002
(86) PCT No.: PCT/JP02/05584

(30) Foreign Application Priority Data
Jun. 8, 2001 (JP) 2001-173983

(51) Int. Cl. A61K 31/4172; A61K 31/401; A61K 31/405; A61K 31/198

(52) U.S. Cl. 514/400; 514/561; 514/562; 514/419; 514/565; 514/566; 514/567; 514/423

(57) ABSTRACT

The present invention forms a mixture basically consisting of threonine, proline, glycine, valine, isoleucine, leucine, tyrosine, phenylalanine, lysine, aspartic acid, serine, glutamic acid, alanine, methionine, histidine and arginine as a unit, by which a body temperature raising effect was observed. The similar body temperature raising effect was observed when carnitine and/or tryptophan is added to the unit. These groups were found to be amino acid mixture-containing agents capable of raising body temperature for food or drink use or medical use. The agents have excellent body temperature raising effect, thereby having the functions of reduction of accumulated body fat, alleviation of excess sensitivity to cold, warming of a cold body, and the like.

The present invention relates to amino acid mixture-containing agents capable of raising body temperature for food or drink use or medical use.

### TECHNICAL FIELD TO WHICH THE INVENTION BELONGS

The present invention relates to amino acid compositions and amino acid composition solutions made up of specified amino acid constituents. The amino acid composition and the solution thereof of the present invention have excellent physiological functions, particularly a body temperature raising function. Besides raising body temperature, it stimulates metabolism. Thus they are effective for improvement of basal metabolism, reduction of body fat accumulation and reduction of excess sensitivity to cold as well as being a means to warm a cold body in cold regions.

### BACKGROUND ART

Conventionally, beverages have been known whose active components are capsaicins (JP-A-2000-189121) utilizing the function of the pungent spice component capsaicin to facilitate energy metabolism.

### PROBLEMS THAT THE INVENTION IS TO SOLVE

Capsaicins are pungent components contained in peppers. The amount included in one dose has been limited since pungency is strongly felt when large amounts of capsaicins are added. In order to warm the body, a method in which hot food and drink are taken and heat is thus incorporated in the body is also feasible, but this effect is transient. Thus, as a composition having a function to improve energy metabolism, it is required that this effect is rapidly obtained and is sustained for some time thereafter. Also, an excellent taste and the like are necessary in the case of oral ingestion.

### MEANS FOR SOLVING THE PROBLEMS

In order to achieve the above objects, the present inventors investigated large numbers of amino acid compositions which were seen to be promising after looking at this problem from various angles. A great number of amino acid compositions were prepared by changing types, combinations, and amounts of amino acids, and their physiological functions were studied in detail. As a result, the present inventors have found for the first time that an amino acid composition with a particular constitution raises basal body temperature and that this effect is sustained for a long time. As the result of further study, the present invention has been completed based on these useful findings.

### BRIEF DESCRIPTION OF THE DRAWINGS

FIG. 1 is a graph showing the average rise in the basal body temperature at each time course when the amino acid composition of the present invention (16 kinds of amino acids in Table 1) was given to rats at each dose.

FIG. 2 is a graph showing the time courses of average basal body temperature when the amino acid composition of the present invention (16 kinds of amino acids in Table 1) was given to rats at each dose.

FIG. 3 is a graph showing the average maximum basal body temperature elevation when the amino acid composition of the present invention (17 kinds of amino acids in Table 4) was given to rats at each dose. Student's t-test was employed for statistical analysis of the experimental results.

FIG. 4 is a graph showing average rise in the basal body temperature at each time course when the amino acid composition of the present invention (17 kinds of amino acids in Table 4) was given to rats. Student's t-test was employed for statistical analysis of the experimental results.

FIG. 5 is a graph showing the time course of average basal body temperature when the amino acid composition of the present invention (17 kinds of amino acids in Table 4) was given to rats.

The present invention is described below.

The first amino acid mixture-containing composition of the present invention consists of threonine, proline, glycine, valine, isoleucine, leucine, tyrosine, phenylalanine, lysine, aspartic acid, serine, glutamic acid, alanine, methionine, histidine and arginine. These 16 amino acids as one mixture constitutes the basic active ingredient of the agent capable of raising body temperature of the present invention.

The second amino acid mixture-containing composition of the present invention consists of threonine, proline, glycine, valine, isoleucine, leucine tyrosine, phenylalanine, lysine, aspartic acid, serine, glutamic acid, alanine, methionine, histidine, arginine and carnitine. Carnitine is regarded here as one of amino acids.

The third amino acid mixture-containing composition of the present invention consists of threonine, proline, glycine, valine, isoleucine, leucine, tyrosine, phenylalanine, lysine, aspartic acid, serine, glutamic acid, alanine, methionine, histidine, arginine and tryptophan.

The fourth amino acid mixture-containing composition of the present invention consists of threonine, proline, glycine, valine, isoleucine, leucine, tyrosine, phenylalanine, lysine, aspartic acid, serine, glutamic acid, alanine, methionine, histidine, arginine, tryptophan and carnitine.

The first amino acid mixture-containing composition of the present invention contains 16 kinds of amino acids as essential components. It is suitable that the respective amounts of each amino acid are in a certain ratio, as follows. It is preferred to have a ratio of from 2 to 15 moles of threonine, from 4 to 30 moles of proline, from 7 to 20 moles of glycine, from 4 to 8 moles of valine, from 3 to 9 moles of isoleucine, from 2 to 12 moles of leucine, from 1 to 9 moles of tyrosine, from 0.5 to 5 miles of phenylalanine, from 5 to 11 moles of lysine, from 0.1 to 5 moles of aspartic acid, from 0.1 to 5 moles of serine, from 0.1 to 4 moles of glutamic acid, from 0.1 to 12 moles of alanine, from 0.1 to 5 moles of methionine, from 0.1 to 5 moles of histidine and from 0.1 to 5 moles of arginine.

The second amino acid mixture-containing composition of the present invention contains 17 kinds of amino acids as essential components. It is suitable that the respective amounts of each amino acid are in a certain ratio, as follows. It is preferred to have a ratio of from 2 to 15 moles of threonine, from 4 to 30 moles of proline, from 7 to 20 moles of glycine, from 4 to 8 moles of valine, from 3 to 9 moles of isoleucine, from 2 to 12 moles of leucine, from 1 to 9 moles of tyrosine, from 0.5 to 5 miles of phenylalanine, from 5 to 11 moles of lysine, from 0.1 to 5 moles of aspartic acid, from 0.1 to 5 moles of serine, from 0.1 to 4 moles of glutamic acid, from 0.1 to 12 moles of alanine, from 0.1 to 5 moles of methionine, from 0.1 to 5 moles of histidine, from 0.1 to 5 moles of arginine and from 0.1 to 5 moles of carnitine.

The third amino acid mixture-containing composition of the present invention contains 17 kinds of amino acids as essential components. It is suitable that the respective amounts of each amino acid are in a certain ratio, as follows. It is preferred to have a ratio of from 2 to 15 moles of threonine, from 4 to 30 moles of proline, from 7 to 20 moles of glycine, from 4 to 8 moles of valine, from 3 to 9 moles of isoleucine, from 2 to 12 moles of leucine, from 1 to 9 moles of tyrosine, from 0.5 to 5 miles of phenylalanine, from 5 to 11 moles of lysine, from 0.1 to 5 moles of aspartic acid, from 0.1 to 5 moles of serine, from 0.1 to 4 moles of glutamic acid, from 0.1 to 12 moles of alanine, from 0.1 to 5 moles of methionine, from 0. 1 to 5 moles of histidine, from 0.1 to 5 moles of arginine and from 0.1 to 5 moles of tryptophan.

The fourth amino acid mixture-containing composition of the present invention contains 18 kinds of amino acids as essential components. It is suitable that the respective amounts of each amino acid are in a certain ratio, as follows. It is preferred to have a ratio of from 2 to 15 moles of threonine, from 4 to 30 moles of proline, from 7 to 20 moles of glycine, from 4 to 8 moles of valine, from 3 to 9 moles of isoleucine, from 2 to 12 moles of leucine, from 1 to 9 moles of tyrosine, from 0.5 to 5 miles of phenylalanine, from 5 to 11 moles of lysine, from 0.1 to 5 moles of aspartic acid, from 0.1 to 5 moles of serine, from 0.1 to 4 moles of glutamic acid, from 0.1 to 12 moles of alanine, from 0.1 to 5 moles of methionine, from 0.1 to 5 moles of histidine, from 0.1 to 5 moles of arginine, from 0.1 to 5 moles of tryptophan and from 0.1 to 5 moles of carnitine.

The amino acid mixture-containing agents capable of raising body temperature for food or drink use or medical use of the present invention are those in which one of the above first to fourth amino acid mixture-containing compositions is the active ingredient. Each amino acid may be free, or a pharmaceutically or food scientifically acceptable salt. Representative salts include hydrochloride and lactate, but these are only examples and the present invention is not limited to these salts. Amino acids each may be orally ingested as such in a mixed powder or an aqueous solution form. However, all of the amino acids have bad taste and are difficult to swallow in either liquid or solid form. Thus, it is better to make them tasty so as to be easily drunk or to make them all into a biscuit form to be easily eaten by using an additive acceptable for food or drink use or medical use together with the active ingredient (from the first to the fourth amino acid mixtures). For example, an easily drinkable preparation can be made by adding an acidifier such as citric acid and a sweetener such as sugar to the aqueous solution of the active ingredient. Also, if refined starch or the like is added to and mixed with the active ingredient in mixed powder form and the mixture is baked into a biscuit form, it can be easily eaten.

As mentioned above, the amino acid mixture-containing agents capable of raising body temperature for food or drink use or medical use of the present invention may be ingested as is in powder form, or dissolved to form an aqueous solution and then ingested. The ingestion method may be any of the common routes of administration such as oral administration, rectal administration, intravenous injection, and drip infusion.

In the case of oral administration, they may be used as powder, granule, tablet, capsule, troche, and the like by mixing with a pharmaceutically acceptable carrier, excipient, or diluent in addition to making them easily drunk or eaten as foods. However, a long time is sometimes required for absorption of solid powder and tablet agents, and thus oral administration of the active ingredient itself is desirable. In this case, the active component may be administered in the aforementioned solution along with appropriate additives, for example, salts such as sodium chloride, a pH adjuster and a chelating agent. In the case of using as an injectable agent, it is better to use those in which an appropriate buffer or isotonic solution is added to the active ingredient, which is dissolved in sterile distilled water.

The ingestion period is not particularly limited. It may be ingested at any suitable time, and is suitable for ingestion, for example, as a drink (including those for medical use in addition to ordinary types such as soft drinks and powder for drinks).

The dosage of the amino acid mixture-containing agents capable of raising body temperature for food or drink use or medical use of the present invention can be set within a wide range. The amount of the active ingredient, usually the amount of from 0.5 to 5 g per dose, preferably from 1 to 3 g per dose, and from 1 to 20 g per day, preferably from 3 to 10 g per day, is determined depending on the administration method or the objective of the use. In the case of administering a solution, from 10 to 1000 ml per dose of a solution 0.5 to 10% by weight, and preferably from 100 to 400 ml per dose of a solution of from 1 to 4% by weight is administered.

As is shown in the examples described below, the amino acid mixture-containing agents capable of raising body temperature for food or drink use or medical use of the present invention have excellent capacity to raise basal body temperature. In conjunction with this, they increase basal metabolism and daily energy consumption. Thus, they are effective for reduction of body fat accumulation and excess sensitivity to cold. Therefore, the present amino acid compositions can be made into various medicines such as body temperature raising agents, anti-cold sensitivity agents by formulating them for medical use.

The amino acid mixture-containing agents capable of raising body temperature for food or drink use or medical use of the present invention have highly effective body temperature raising function. They may be used in solution as mentioned above, particularly in aqueous solution, in addition the original powder form. In the case of the solution, the composition of the present invention may be dissolved as is in water to prepare the solution, or each amino acid may be separately dissolved in water to realize the aforementioned constitution in the solution.

The examples of the present invention are described below.

### EXAMPLE 1

(1) Method

Four groups of 8- to 9-weeks old Crj:CD(SD)IGS male rats with 4 rats per group were prepared, and were used for the experiments after overnight fasting.

The first group was control (saline). For the second group 0.2 g of the amino acid composition of the invention per 100 g of body weight (2 g/kg), 0.1 g per 100 g of body weight (1 g/kg) for the third group, and 0.05 g per 100 g of body weight (0.5 g/kg) for the fourth group were administered once intraperitoneally. The amino acid composition of the invention (the composition having 16 kinds of amino acids in the given constitution) was suspended in 0.8 ml of saline per 100 g of body weight (8 ml/kg). For the control group, 0.8 ml of saline (Hikari Seiyaku KK, Pharmacopoeia of Japan saline solution, Lot. 9911HC) per 100 g of body weight (8 ml/kg) alone was administered.

(2) Substance to be Tested

The amino acid composition shown in the following Table 1 is one embodiment of the composition of the invention. All amino acids were obtained from Wako Pure Chemical.

TABLE 1

| Amino acid | mole % |
|---|---|
| Histidine | 2.6 |
| Arginine | 3.6 |
| Isoleucine | 4.6 |
| Leucine | 6.3 |
| Lysine hydrochloride | 8.8 |
| Lysine hydrochloride | 8.8 |
| Phenylalanine | 3.9 |
| Tyrosine | 6.1 |
| Valine | 6.0 |
| Aspartic acid | 0.2 |
| Serine | 2.5 |
| Glutamic acid | 3.3 |
| Proline | 18.4 |
| Glycine | 19.5 |
| Alanine | 6.3 |
| Threonine | 7.4 |
| Methionine | 0.5 |

(3) Administration and Measurement of Body Temperature

The substance to be tested was administered using an aliform needle for intravenous injection. In order to confirm that the drug was surely administered intraperitoneally, a dye was administered after the measured dose was given to confirm that the subject of testing was not administered into the intestine.

The animal was laid on the back under anesthesia with urethane (1.2 g/kg, i.p.), and the aliform needle for intravenous injection was kept in the peritoneal cavity for intraperitoneal administration. A probe for body temperature measurement was inserted from the anus into rectum and fixed. The body temperature was measured every 10 min. The temperature at 0 min after the administration was the basis. The basal body temperatures 10 min before, after 10 min, after 20 min, . . . and so on up to after 120 min were measured, and the average value was calculated from the results of each time to make tables and figures (graphs).

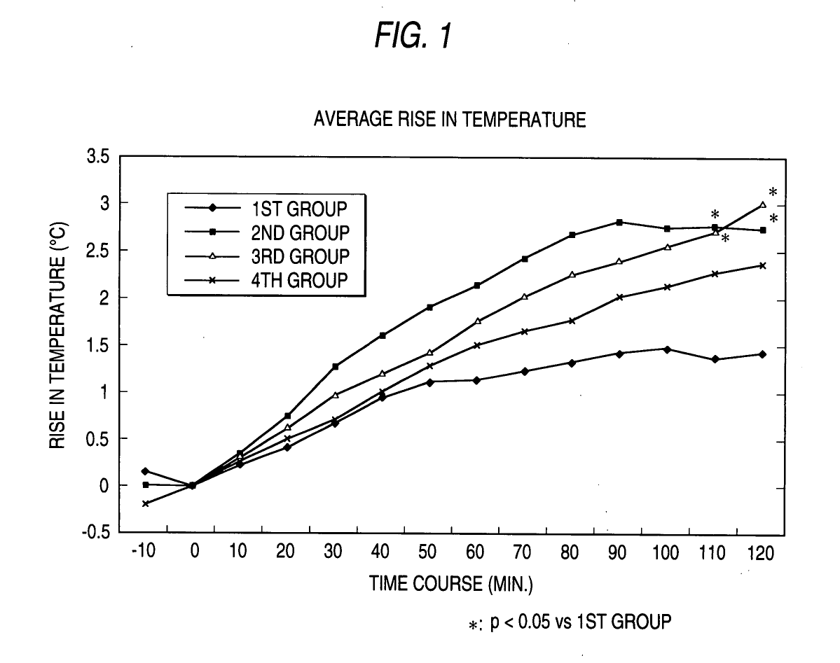

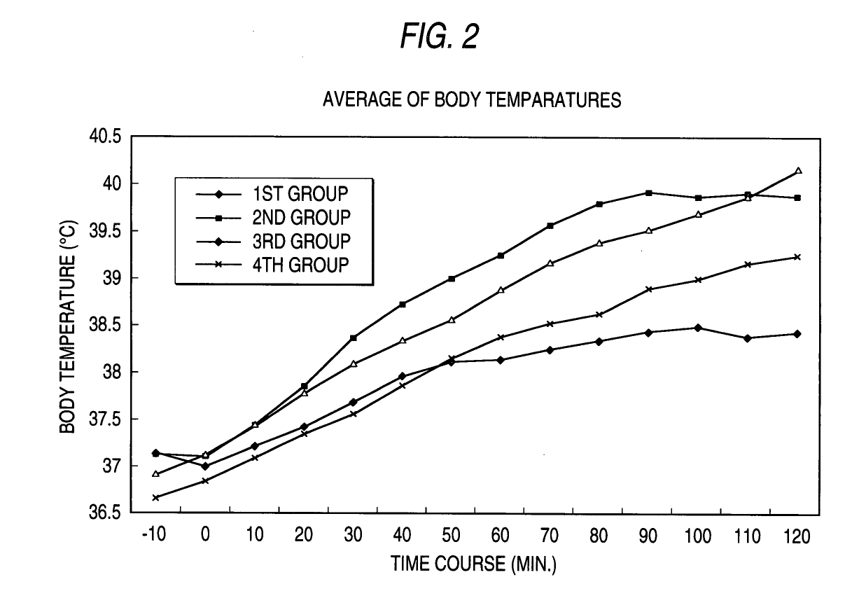

That is, the average value of the elevated temperature of the basal body temperature at each time course was shown in the following Table 2 and FIG. 1. The average of the basal body temperature at each time course is shown in the following Table 3 and FIG. 2.

TABLE 2
Average of elevated temperature of
basal body temperature at each time course

| Minute | −10 | 0 | 10 | 20 | 30 | 40 | 50 |
|---|---|---|---|---|---|---|---|
| 1st Group | 0.15 | 0 | 0.225 | 0.425 | 0.675 | 0.95 | 1.1 |
| 2nd Group | 0.025 | 0 | 0.35 | 0.75 | 1.275 | 1.6 | 1.9 |
| 3rd Group | −0.2 | 0 | 0.325 | 0.65 | 0.975 | 1.2 | 1.425 |
| 4th Group | −0.2 | 0 | 0.25 | 0.5 | 0.7 | 1 | 1.275 |
| | 60 | 70 | 80 | 90 | 100 | 110 | 120 |
| | 1.125 | 1.225 | 1.325 | 1.425 | 1.475 | 1.375 | 1.425 |
| | 2.125 | 2.425 | 2.675 | 2.8 | 2.75 | 2.775* | 2.75* |
| | 1.75 | 2.025 | 2.25 | 2.375 | 2.55 | 2.725* | 3.025* |
| | 1.5 | 1.65 | 1.75 | 2.025 | 2.125 | 2.275 | 2.375 |

*p < 0.05 versus 1st Group

TABLE 3
Average of basal body temperature at each time course

| Minute | −10 | 0 | 10 | 20 | 30 | 40 | 50 |
|---|---|---|---|---|---|---|---|
| 1st Group | 37.15 | 37 | 37.225 | 37.425 | 37.675 | 37.95 | 38.1 |
| 2nd Group | 37.125 | 37.1 | 37.45 | 37.85 | 38.375 | 38.7 | 39 |
| 3rd Group | 36.925 | 37.125 | 37.45 | 37.775 | 38.1 | 38.325 | 38.55 |
| 4th Group | 36.65 | 36.85 | 37.1 | 37.35 | 37.55 | 37.85 | 38.125 |
| | 60 | 70 | 80 | 90 | 100 | 110 | 120 |
| | 38.125 | 38.225 | 38.325 | 38.425 | 38.475 | 38.375 | 38.425 |
| | 39.225 | 39.525 | 39.775 | 39.9 | 39.85 | 39.875 | 39.85 |
| | 38.875 | 39.15 | 39.375 | 39.5 | 39.675 | 39.85 | 40.15 |
| | 38.35 | 38.5 | 38.6 | 38.875 | 38.975 | 39.125 | 39.225 |

(4) Results and Discussion

As is clearly shown in the above results, the levels of the body temperature in the control group were slightly increased. The dose-dependent elevation of the body temperature was demonstrated in the groups given the substance to be tested. A significant difference was observed in the 2nd and 3rd groups compared to the control group.

### EXAMPLE 2

(1) Method

Two groups of 8- to 10-weeks old SD male rats and six per group were prepared. After overnight fasting, they were used for the experiments. The grouping was carried out depending on the body weight on the same day.

(2) Substance to be Tested

As the substance to be tested, 17 kinds of amino acids (VAAM) shown in Table 4 were used. The formulated ratios were as is shown in Table 4. All amino acids were obtained from Wako Pure Chemicals Co., Ltd.

TABLE 4
Seventeen kinds of amino acids: VAAM

| Amino acid | Component weight (%) | mole (%) |
|---|---|---|
| Histidine | 3.2 | 2.6 |
| Arginine | 4.9 | 3.5 |
| Isoleucine | 4.7 | 4.5 |
| Leucine | 6.4 | 6.1 |
| Lysine hydrochloride | 12.5 | 8.6 |
| Phenylalanine | 5.0 | 3.8 |
| Tyrosine | 8.6 | 6.0 |
| Valine | 5.5 | 5.9 |
| Aspartic acid | 0.2 | 0.2 |
| Serine | 2.1 | 2.5 |
| Glutamic acid | 3.7 | 3.2 |
| Proline | 16.5 | 18.0 |
| Glycine | 11.4 | 19.1 |
| Alanine | 4.3 | 6.1 |
| Threonine | 6.8 | 7.2 |
| Methionine | 0.6 | 0.5 |
| Tryptophan | 3.6 | 2.2 |

(3) Dosage

The first group was the control (injectable water). The rats in the second group were once administered orally 0.5 g/100 g body weight (5 g/kg) of 17 kinds of amino acids.

The substance to be tested of the amino acids was suspended in 1 ml/100 g body weight (10 ml/kg) of saline. The control animals were given 1 ml/100 g body weight (10 ml/kg) of saline solution (Hikari Seiyaku KK, Pharmacopoeia of Japan saline solution, Lot. 9911HC).

(4) Temperature Measurement

The animals were placed and positioned in Bollman cages. The probe for measuring the body temperature was inserted from the anus into rectum and fixed. The body temperature was measured every 10 to 15 min. When the levels of the body temperature became stable, the substance to be tested was administered. Then, using the temperature at 0 min after the ingestion as the standard, the body temperature was measured every 10 min until 90 min after the ingestion. The average values were calculated from the results in each group and shown in the graphs. The maximally elevated body temperatures were averaged in each group, and the differences between groups were shown.

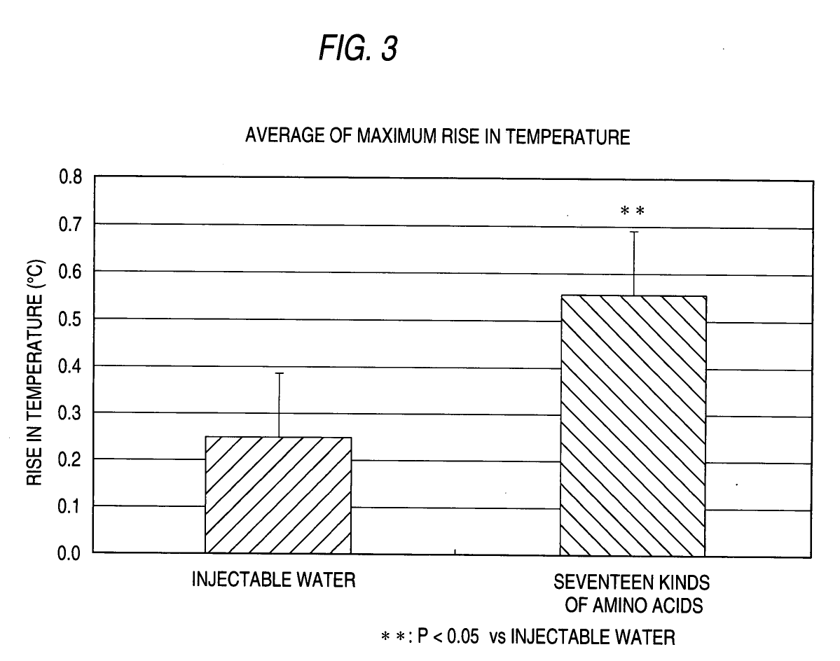

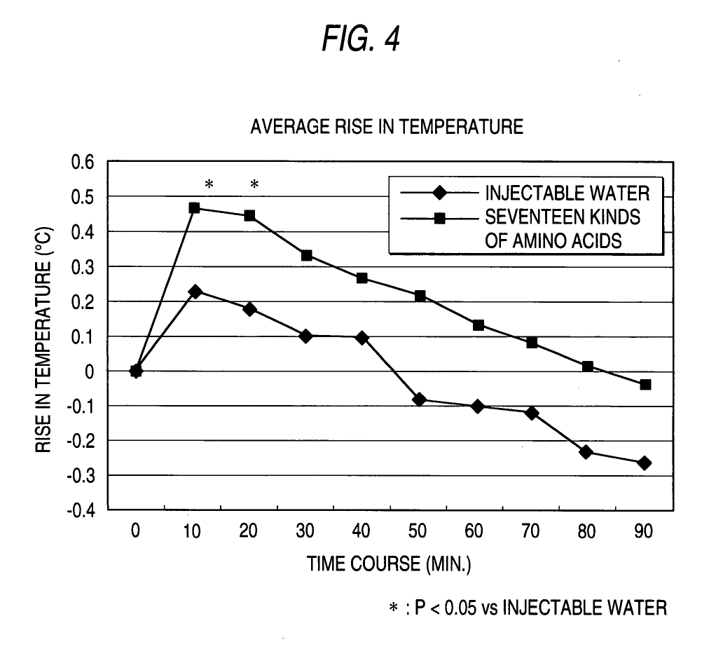

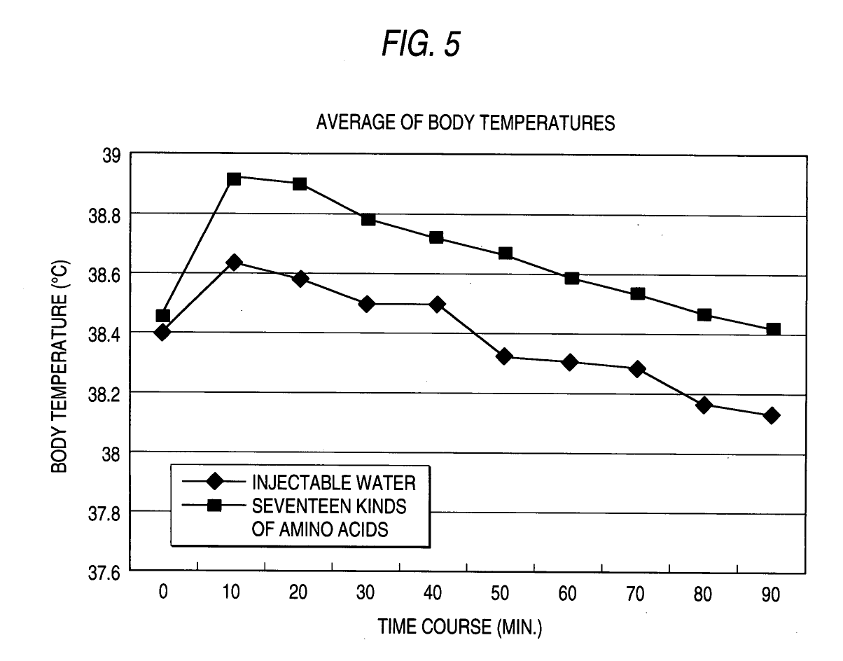

For 17 kinds of amino acids, the average values of the maximally elevated body temperatures were shown in FIG. 3, and the average values of the increment of the body temperatures and body temperatures per se at each time course are shown in FIGS. 4 and 5, respectively.

(5) Results and Discussion

As is clearly shown in the above results, the levels of the temperature in the control group were slightly increased over the time course. The animals in the group given the substance to be tested (17 kinds of amino acids: VAAM) exhibited the excellent elevation of the body temperature.

Effect of the Invention

The present invention has found that an active ingredient made up of certain amino acids has a novel use—an excellent action for elevation of body temperature. By administering the amino acid mixture-containing agents of the invention capable of raising body temperature for food or drink use or medical use, everyday body temperature is elevated, resulting in increased energy consumption and high basal metabolism. They are also effective for the elevation in basal body temperature, the reduction in body fat accumulation and alleviation of excess sensitivity to cold, as well as for warming the body when it is cold.

1. An amino acid mixture-containing agent capable of raising body temperature for food or drink use or medical use, which comprises, as an active ingredient, a group consisting of threonine, proline, glycine, valine, isoleucine, leucine, tyrosine, phenylalanine, lysine, aspartic acid, serine, glutamic acid, alanine, methionine, histidine and arginine.

2. An amino acid mixture-containing agent capable of raising body temperature for food or drink use or medical use, which comprises, as an active ingredient, a group consisting of threonine, proline, glycine, valine, isoleucine, leucine, tyrosine, phenylalanine, lysine, aspartic acid, serine, glutamic acid, alanine, methionine, histidine, arginine and carnitine.

3. An amino acid mixture-containing agent capable of raising body temperature for food or drink use or medical use, which comprises, as an active ingredient, a group consisting of threonine, proline, glycine, valine, isoleucine, leucine, tyrosine, phenylalanine, lysine, aspartic acid, serine, glutamic acid, alanine, methionine, histidine, arginine and tryptophan.

4. An amino acid mixture-containing agent capable of raising body temperature for food or drink use or medical use, which comprises as an active ingredient, a group consisting of threonine, proline, glycine, valine, isoleucine, leucine, tyrosine, phenylalanine, lysine, aspartic acid, serine, glutamic acid, alanine, methionine, histidine, arginine, tryptophan and carnitine.

5. An amino acid mixture-containing agent capable of raising body temperature for food or drink use or medical use, which consists of the active ingredient according to any one of claims 1 to 4 and an additive acceptable for food or drink use or medical use.

6. The amino acid mixture-containing agent capable of raising body temperature for food or drink use or medical use according to claim 1, wherein the ratio of the amino acids is from 2 to 15 moles of threonine, from 4 to 30 moles of proline, from 7 to 20 moles of glycine, from 4 to 8 moles of valine, from 3 to 9 moles of isoleucine, from 2 to 12 moles of leucine, from 1 to 9 moles of tyrosine, from 0.5 to 5 moles of phenylalanine, from 5 to 11 moles of lysine, from 0.1 to 5 moles of aspartic acid, from 0.1 to 5 moles of serine, from 0.1 to 4 moles of glutamic acid, from 0.1 to 12 moles of alanine, from 0.1 to 5 moles of methionine, from 0.1 to 5 moles of histidine and from 0.1 to 5 moles of arginine.

7. The amino acid mixture-containing agent capable of raising body temperature for food or drink use or medical use according to claim 2, wherein the ratio of the amino acids is from 2 to 15 moles of threonine, from 4 to 30 moles of proline, from 7 to 20 moles of glycine, from 4 to 8 moles of valine, from 3 to 9 moles of isoleucine, from 2 to 12 moles of leucine, from 1 to 9 moles of tyrosine, from 0.5 to 5 moles of phenylalanine, from 5 to 11 moles of lysine, from 0.1 to 5 moles of aspartic acid, from 0.1 to 5 moles of serine, from 0.1 to 4 moles of glutamic acid, from 0.1 to 12 moles of alanine, from 0.1 to 5 moles of methionine, from 0.1 to 5 moles of histidine, from 0.1 to 5 moles of arginine and from 0.1 to 5 moles of carnitine.

8. The amino acid mixture-containing agent capable of raising body temperature for food or drink use or medical use according to claim 3, wherein the ratio of the amino acids is from 2 to 15 moles of threonine, from 4 to 30 moles of proline, from 7 to 20 moles of glycine, from 4 to 8 moles of valine, from 3 to 9 moles of isoleucine, from 2 to 12 moles of leucine, from 1 to 9 moles of tyrosine, from 0.5 to 5 moles of phenylalanine, from 5 to 11 moles of lysine, from 0.1 to 5 moles of aspartic acid, from 0.1 to 5 moles of serine, from 0.1 to 4 moles of glutamic acid, from 0.1 to 12 moles of alanine, from 0.1 to 5 moles of methionine, from 0.1 to 5 moles of histidine, from 0.1 to 5 moles of arginine and from 0.1 to 5 moles of tryptophan.

9. The amino acid mixture-containing agent capable of raising body temperature for food or drink use or medical use according to claim 4, wherein the ratio of the amino acids is from 2 to 15 moles of threonine, from 4 to 30 moles of proline, from 7 to 20 moles of glycine, from 4 to 8 moles of valine, from 3 to 9 moles of isoleucine, from 2 to 12 moles of leucine, from 1 to 9 moles of tyrosine, from 0.5 to 5 moles of phenylalanine, from 5 to 11 moles of lysine, from 0.1 to 5 moles of aspartic acid, from 0.1 to 5 moles of serine, from 0.1 to 4 moles of glutamic acid, from 0.1 to 12 moles of alanine, from 0.1 to 5 moles of methionine, from 0.1 to 5 moles of histidine, from 0.1 to 5 moles of arginine, from 0.1 to 5 moles of tryptophan and from 0.1 to 5 moles of carnitine.

10. An amino acid mixture-containing agent capable of raising body temperature for food or drink use or medical uses, which comprises the active ingredient according to any of claims 6 to 9 and an additive acceptable for food or drink use or medical use.

11. A method for raising body temperature, which comprises administering the agent according to any one of claims 1 to 4 and 6 to 9.

12. The method according to claim 11, wherein the agent contains an additive acceptable for food or drink use or medical use.
